(19) United States
(12) Patent Application Publication
(10) Pub. No.: US 2005/0216036 A1
(43) Pub. Date: Sep. 29, 2005

(54) Endoscopic fastening system with multiple fasteners

(76) Inventor: Naomi L. Nakao, New York, NY (US)

Correspondence Address: R. Neil Sudol, 714 Colorado Avenue, Bridgeport, CT, 06605-1601, US

(21) Appl. No.: 11/090,718

(22) Filed: Mar. 25, 2005

Related U.S. Application Data
(60) Provisional application No. 60/557,201, filed on Mar. 29, 2004.
(60) Provisional application No. 60/611,257, filed on Sep. 17, 2004.

(51) Int. Cl. A61B 17/10

(52) U.S. Cl. 606/142; 606/139

(57) ABSTRACT

An endoscopic fastening system having a diameter sufficiently small so that the fastening system is slidably insertable into a working channel of a flexible or rigid endoscope for performing a stapling, clipping, or other fastening operation, comprises one or more fasteners, and a fastener delivery and deployment assembly. One or more fasteners being disposed in a substantially closed prefiring configuration inside the fastener delivery and deployment system are configured for being delivered and deployed into a target tissue while remaining in a substantially closed configuration throughout the entire operation. Other related embodiments of fasteners and their respective delivery and deployment assemblies enabling fastening operations in conjunction with a flexible or rigid endoscope are described.

### CROSS-REFERENCE TO RELATED APPLICATIONS

This application claims the benefit of the priority of U.S Provisional Application No. 60/557,201, filed on Mar. 29, 2004, and Provisional Application No. 60/611,257, filed on filed Sep. 17, 2004.

### FIELD OF THE INVENTION

The present invention relates to an endoscopic fastening device and surgical fasteners to be used in conjunction with flexible or rigid endoscopy, or during open surgery. Fastening is performed on internal body tissues as part of a surgical diagnostic or therapeutic procedure using one or more surgical fasteners and an associated fastener delivery and deployment assembly designed for delivering one or more fasteners without having to remove the device from the body after each fastener application.

### BACKGROUND OF THE INVENTION

Conventional surgical techniques for treating medical anomalies in the gastrointestinal tract often necessitate the use of fasteners such as, for example, staples, clips etc. Such procedures generally require that an extensive incision be made (open surgery), or that a series of small incisions be created, through which several cannulas are placed for providing access to a body cavity (laparoscopic surgery).

Currently, there is no reliable method for securing fasteners inside a patient's body in conjunction with a flexible endoscope. The challenge entailed in creating a fastener delivery and deployment device that may be passed through a flexible endoscope is two-fold: firstly, the working channel of an endoscope is very narrow requiring a device with an outer diameter sufficiently small to pass through said working channel. Secondly, a flexible endoscope bends along with curvatures present, for example, in the gastrointestinal tract, requiring similar flexibility of a fastener delivery and deployment device to enable such a journey. Both of these challenges have not yet been surmounted, hence there are no such devices currently available.

Suturing operations have the same limitations, and as such physicians have been unable to perform surgical procedures via natural body orifices using a flexible endoscope. Surgical procedures to remove diseased tissue or organs such as colon resection, gall bladder removal, or stomach resection are currently being performed via open or laparoscopic surgery. In addition, surgery for morbid obesity (bariatric surgery), which is being performed with much greater frequency, also requires either an open or laparoscopic technique.

Laparoscopic surgery has been developing rapidly in the past few years because it is less invasive than open surgery. These procedures enable sewing or stapling tissue via a series of small abdominal incisions through which a number of cannulas are placed. Rigid instruments are passed through these cannulas and manipulated from outside the body. The surgical procedure is visualized with a camera, which is introduced through a separate cannula.

Providing smaller diameter instruments capable of reaching surgical sites through smaller access ports or cannulas would provide an advantage during laparoscopic surgery because smaller incisions cause a lesser injury, providing for a more rapid healing process. The size of the instruments used to deliver surgical fasteners, such as for example staples, is dictated by fastener size. If the fasteners were to be passed into the body in an open configuration, a larger diameter fastener delivery device would be required. Such a delivery device would be too large for minimally invasive operations.

The currently used fastener delivery device sizes have been decreased by designing the device for delivery of a closed staple. This enables passage of these devices through smaller diameter cannulas. Upon reaching the operative site, such a fastener must be opened by some means, in order to engage a target tissue, after which the fastener is again closed upon the tissue (closed-open-closed design).

A fastener delivery device used during laparoscopic surgery is, by necessity long and slender, its distal working end being far removed from the operating handle outside a patient's body. Consequently, a staple may be displaced, or slip out of the delivery device's jaws. Furthermore, the force required to open and close the fastener is magnified because it is transmitted through the distance of the shaft. Providing a fastening system, that would permit introduction and delivery of a fastener in a substantially closed configuration, would preclude a need for the “closed open closed” design.

Certain procedures require that a surgeon ligate or close various tubular structures, such as blood vessels or fluid ducts prior to severing them. In order to ligate a tubular structure, it must first be clamped using a clamping device. This procedure is followed by clip application, using a second device. In the event that the tubular structure is small, and particularly when it is embedded in other tissue structures, it may be difficult to place both the clamping and clipping devices onto this small tubular structure. Providing a fastener delivery and deployment device that clamps and clips using the same instrument, saves a step, precludes using an extra instrument, and enables more precise clip placement.

Excessive bleeding occurs during a surgical procedure when an artery is inadvertently severed. When such an event occurs during laparoscopic surgery, the problem requires “long distance” management, which entails placement of clamps passed through cannulas directed towards the bleeding site. Once hemostasis (cessation of bleeding) is achieved by the clamping instrument, a staple or clip delivery device is introduced through a second cannula, and a clip is applied at a point adjacent to the clamp.

It is frequently impossible to see the clip placement, as it may be embedded in tissue. If the clip were not placed correctly, bleeding resumes upon release of the clamping device, requiring that the entire procedure be repeated. In the interim, dangerous blood loss may occur, especially if the severed vessel is an artery. Providing a device which clamps and clips in the same maneuver would enable clip placement through the very same instrument that provides clamping, thus ensuring correct clip placement, and prompt hemostasis.

While laparoscopic procedures are less invasive than open surgery, they are often more lengthy requiring extensive general anesthesia, and prolonged periods of convalescence. A means to provide stapling or clipping in conjunction with a flexible endoscope would reduce the length and complexity of surgery. Because flexible endoscopic procedures are typically performed under conscious sedation and are less invasive, they are naturally less traumatic to the body. Convalescence is significantly shortened, postoperative pain virtually eliminated and patients are ambulatory within hours after an endoscopic procedure.

Although there appear to be no commercial devices on the market that enable stapling through the working channel of the flexible endoscope, U.S. Pat. Nos. 5,222,961, 5,156,609, 5,015,249 and 5,049,153 to Nakao et al describe various embodiments of an endoscopic stapling device. Patent number 5,015,249 describes a flexible stapler whereby the staple is configured with an open bias, and releasably connected to a rod member. The staple is ejected by pushing the rod member forward. Upon engagement of the staple with tissue, the staple being opened by its open bias, a tubular member is pushed over the staple to close it. The problem with the embodiment of the '249 patent is the following: bowel wall thickness, for example, is approximately 05 cm, and its consistency is slightly firmer than that of a calf's liver. Closing an indwelling staple by pushing a tubular member over it, may push the entire staple through the bowel wall, thus causing a perforation.

U.S. Pat. No. 5,049,153 describes a flexible stapler, wherein a staple with an open bias is disposed in the prefiring position inside said stapler's open jaws. The stapler is brought to the tissue with the indwelling staple, closed upon the tissue, and once the staple locks, the staple legs are released. The staple described by the '153 patent is a spring biased staple with an open bias. The invention described herein below does not employ such a staple.

U.S. Pat. No. 5,156,609 describes a plurality of second staples each having a spring bias.

U.S. Pat. No. 5,222,961 describes various additional means of locking a staple.

It is therefore desirable to provide a surgical fastener with associated delivery and deployment assembly for applying one or more fasteners and for providing tissue clamping, said fastener delivery and deployment assembly being configured for passing through the working channel of a flexible endoscope.

It is further desirable to provide a fastening system capable of reaching surgical sites through smaller access ports during laparoscopic surgery, said device being configured for clamping target tissue, as well as fastener delivery and application,

It is yet further desirable to provide a fastener delivery system whereby the fastener may remain in a substantially closed configuration throughout the entire fastening operation, said fastening system configured for passing through the working channel of a flexible endoscope or laparoscope.

The benefits of the present invention in addressing the drawbacks of the prior art and the objectives and needs noted above will be more readily apparent from the description and drawings of the invention set forth herein.

### DEFINITIONS

The term “endoscopic” is used herein to designate any of a variety of minimally invasive surgical procedures wherein optical elements are used to view internal spaces and tissues of a patient through relatively small surgically created openings or natural orifices. Concomitantly, the term “endoscope” as used herein refers to any optical or tubular instrument inserted through such openings or orifices for purposes of enabling visualization of and/or access to internal tissues during a minimally invasive procedure.

During a laparoscopic procedure, for example, an optical element may be inserted through one small incision, while one or more cannulas would be inserted through one or more separate incisions. The surgical instruments inserted through the cannulas are visualized by means of the first optical element. During a flexible endoscopic procedure on the other hand, a flexible endoscope may include, for example, both the optical element and one or more channels through which the surgical instruments are passed.

An endoscopic fastening device as described herein below is inserted through a working channel of an endoscope. As described above, an “endoscope” may include optical illumination and image transmission components or may be a simple tube, such as a cannula. More generally, an endoscope may be any instrument through which an endoscopic diagnostic or minimally invasive surgical procedure is performed.

### BRIEF DESCRIPTION OF THE INVENTION

As broadly contemplated, the endoscopic fastening system of the invention comprises a fastener delivery and deployment assembly and related surgical fasteners for use during flexible or rigid endoscopy, and open surgery. The invention relates to a fastening operation performed on internal body tissues as part of a surgical, diagnostic or therapeutic procedure using one or more surgical fasteners, and an associated fastener delivery and deployment assembly capable of delivering multiple fasteners without removing the device from the body.

In one embodiment of the present invention, an endoscopic fastening system comprises an elongate shaft member having an outer diameter sufficiently small, so that the shaft member may be slidably insertable through a working channel of an endoscope. The working channel may be built into the endoscope insertion member or may be part of an endoscope sheath. The shaft member is provided at a distal end thereof with a pair of forceps jaws, the jaws being configured with preformed grooves extending longitudinally on an inwardly facing surface. An actuator mechanism is provided proximate a proximal end of the shaft member, the actuator being configured for manipulating the forceps jaws between opened and closed positions.

A surgical fastener is provided, comprising two legs and a bight portion joining the first leg to the second leg. The fastener is disposed in a partially closed configuration within an inner channel or lumen of the tubular shaft member. The channel surrounding a fastener maintains the fastener in a partially closed prefiring configuration. A plurality of identical fasteners may be disposed end to end, forming a fastener magazine, contained within the channel of the tubular shaft member.

In a method for performing surgical operations on internal body tissues of a patient, in accordance with the embodiment of present invention described herein above, the following steps are taken: (i) inserting an endoscope through an aperture in the patient's body and locating a target tissue; (ii) pushing a fastener delivery and deployment assembly in a distal direction towards the target tissue. (iii) opening forceps jaws and inserting the jaws into a target tissue; (iv) closing the jaws to a first level of closure or a first closed configuration, thereby causing tissue approximation; and (v) upon achieving the proper approximation, advancing a first fastener in the distal direction into the already closed jaws. (There is no need to force the fastener open prior to its delivery into the jaws, as the fastener slides into the designated preformed grooves, and enters tissue). The method further comprises (vi) squeezing the jaws into an ultimately shut position, thereby permanently deforming the fastener's bight portion for secure fastener closure; and (vii) opening the jaws, thereby releasing the indwelling fastener. The procedure may be repeated several times using some or all fasteners of the magazine.

The above-described method is an important advance in the technology of small fasteners (staples, clips, etc) because it precludes the need for the “opened-closed-opened” technique. This method, described herein for the first time, enables the “closed-closed-closed” technique, thereby simplifying the design of an endoscopic fastening system to the extent of enabling the creation of such a device for use in conjunction with a flexible endoscope.

In another embodiment described herein below in accordance with the invention, an endoscopic fastening system comprises an elongate shaft member having an outer diameter sufficiently small to be slidably insertable through a working channel of an endoscope. The shaft member is provided at a distal end thereof with a pair of forceps jaws and at a proximal end with an actuation mechanism. A surgical fastener is provided, comprising two legs and a bight portion joining the first leg to the second leg. The fastener is disposed in a substantially closed configuration within a channel or lumen of the shaft member, the channel or lumen surrounding a fastener maintaining the fastener in a substantially closed prefiring configuration. A plurality of second fasteners disposed end to end forming a fastener magazine are contained within the channel of the shaft member. A pusher rod member is configured and used to advance fasteners contained within the shaft member towards a distal end of the shaft member. A clevis subassembly is disposed proximate the proximal aspect of the forceps jaws, the clevis subassembly being configured to include a forming rail. The clevis subassembly is operatively coupled with the forceps jaws such that when a fastener advanced into the clevis subassembly towards the direction of the forceps jaws, the fastener is forced from a closed to an open configuration as it traverses the forming rail. A mechanism is provided for advancing further fasteners in a distal direction towards the forceps jaws upon firing of a first fastener, as further described herein below.

In a method for fastening internal body tissues of a patient, in accordance with the present invention, an endoscope is inserted through an aperture in a patient's body and is used to visually locate a target tissue to be operated upon. When a surgical site is localized, a fastener delivery and deployment assembly is inserted through a working channel of the endoscope and advanced in the distal direction towards the target tissue. Upon reaching the target tissue, forceps jaws are opened by manipulating an actuation mechanism, and the jaws are pushed towards the target tissue until the sharpened forceps jaws rest firmly upon the target tissue. A fastener is advanced into the open forceps jaws by ejecting the substantially closed fastener through a forming rail and into the preformed grooves in the jaws, thus forcing the fastener into an open configuration inside the open jaws. The fastener is held securely within the open jaws by means of the preformed rails, and by resting firmly on the target tissue, thus preventing the fastener's displacement from the forceps jaws. The open forceps jaws with the open indwelling fastener are then plunged into the target tissue. An actuation mechanism comprising means for several degrees of closure is activated causing the jaws, and thereby the indwelling fastener, to close upon the target tissue. Once properly placed inside the target tissue the jaws are closed even further, thereby permanently deforming the fastener's bight portion for secure fastener closure. The jaws are then opened to release the fastener and are extracted from the target tissue. The procedure may be repeated several times using some or all of the fasteners of the magazine.

Yet another embodiment of an endoscopic fastening system in accordance with the present invention is described herein below, the embodiment presenting subject matter matter over and beyond U.S Provisional Application No. 60/557,201, filed on Mar. 29, 2004, and U.S. Provisional Application No. 60/611,257, filed on filed Sep. 17, 2004.

A fastening system in accordance with the present invention comprises a delivery and deployment assembly of the embodiment which includes a shaft member having a longitudinal channel of lumen, surgical fasteners disposed in a magazine fashion within the channel or lumen, and a pusher rod member configured and used to advance fasteners contained within the shaft's channel or lumen towards the shaft member's distal end.

A surgical fastener is made of a spring biased material such as plastic or surgical stainless steel, or a shape memory material such as Nitinol (NiTi). The surgical fastener, in accordance with the embodiment of this invention, is provided with a pair of prongs, and with a proximal spring member operably coupled with the prongs, such that when the spring member is squeezed from lateral directions, placing the spring member into a strained state, the fastener prongs assume an open configuration. When the spring member is released, placing the spring member into an unstrained state, the prongs spring back to a closed configuration. A fastener of this embodiment, in accordance with the invention, is disposed in a closed prefiring configuration as it longitudinally traverses the channel of the shaft member.

A shaft member is provided, such that the shaft member includes a collar member made of a hard material, such as, for example, stainless steel, the collar member being configured such that the diameter of its inner lumen is smaller than that of the shaft member's channel, providing a so called “restrictive lumen”. The restrictive lumen may be tapered so as to have a wider proximal opening that is contiguous with the inner tubular member, the opening configured for guiding the distal tips of the fastener into the collar member.

In a method for fastening internal body tissues of a patient, in accordance with the present invention, an endoscope is inserted through an aperture in a patient's body and is used to visually locate a target tissue to be operated upon. When a surgical site is localized, a fastener delivery and deployment assembly is inserted into a working channel of the endoscope and advanced in a distal direction towards a target tissue. Upon reaching the target tissue, a pusher rod member is used to advance fasteners contained in a tubular shaft member towards a collar member. When a most distal fastener's prongs enter into the collar, the prongs being in a closed configuration, the prongs pass through the restrictive lumen without obstruction, because the restrictive lumen's inner diameter is appropriately sized for such passage.

A spring member of the fastener is disposed at a trailing or proximal end thereof and is larger than the inner diameter of the restrictive lumen. When the spring member is advanced into the restrictive lumen, the spring member becomes constricted by virtue of it being larger than the inner diameter of the restrictive lumen. Hence, a compressive force applied to the spring member provides for opening of the prongs into a pre-firing state as discussed herein above.

When the fastener assumes its maximally opened configuration, the fastener is advanced into a target tissue by manipulation of the shaft member, an actuation assembly, or by manipulation of the endoscope, until the fastener enters target tissue, the fastener being in an open, strained state. Upon further advancing the fastener by the pusher rod member, the spring member of the fastener is released from the restrictive lumen, thereby releasing the tension on the spring member, and causing the fastener prongs to snap closed into a postfiring closed configuration within the target tissue.

The above described embodiment may be configured with or without jaws for various surgical operations further described herein below.

In another method for performing a surgical operation on internal body tissues of a patient, in accordance with the present invention, a plurality of surgical fasteners provided with holes or cavities proximate a proximal end of the fasteners are adjoined to one another by a suture thread, the fasteners being preloaded in pairs or in a magazine fashion. The fasteners may be applied in pairs at opposite ends or along opposite edges of a tissue cut, and subsequently synched together to provide tissue closure. Alternatively, the fasteners may be applied in a crisscross fashion into two opposing stomach walls during gastric restriction surgery for morbid obesity. When the first stage of the above fastener application is completed, it simulates an open shoelace. The two loose ends of the suture thread may then be synched together, causing opposite stomach walls to approximate, thus achieving restriction of a gastric pouch.

A surgical fastening instrument with jaws comprising distally rounded tips, the instrument containing a magazine of staples with similarly rounded tips utilizable for ligating tubular structures internal to a patient's body is yet another feature of the present invention. This surgical fastener may be used in conjunction with a flexible or rigid endoscope, during open, laparoscopic or flexible endoscopic procedures, or during open surgery.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a perspective view, partially in cross section, of a distal end of a tissue fastening system in accordance with the present invention, showing the system near an incision or wound in organic tissue.

FIGS. 1A-C depict detailed perspective views of a fastener delivery and deployment assembly (1A), a cut away section of the shaft member of the assembly (1B), and a singular fastener in a partially closed configuration.

FIGS. 1D-F are perspective views of a single forceps jaw (1D), a pusher rod member (1E), and a clevis subassembly (1F), all component members of a fastener delivery and deployment assembly.

FIG. 2 is a perspective view, similar to FIG. 1, showing the distal end of the endoscopic fastening system of FIG. 1 with forceps jaws opened and entering target tissue.

FIG. 3 is a perspective view, similar to FIG. 2, showing the forceps jaws closed upon a target tissue.

FIG. 4 is a perspective view, similar to FIG. 3 but on a smaller scale, showing a surgical fastener being deployed into target tissue within closed forceps jaws.

FIG. 5 is a perspective view, similar to FIG. 4, showing the further closing of the forceps jaws to bring an indwelling surgical fastener into a fully closed postfiring configuration.

FIG. 6 is a perspective view of a deployed surgical fastener, clamped into its fully closed postfiring configuration upon a target tissue in accordance with the invention.

FIGS. 7A-D depict schematic perspective views of a portion of the clevis subassembly of FIG. 1F with a forming rail (7A), a surgical fastener in a partially closed prefiring configuration (7B), a fastener prior to engaging a forming rail (7C), and same fastener having traversed the forming rail, the fastener being forced from a partially closed to an open prefiring configuration (7D).

FIG. 8 is a perspective depiction of two fasteners traveling through a shaft member, entering a clevis subassembly, the distal fastener being forced open by a forming rail.

FIGS. 9-12 illustrate perspective views of an alternative surgical fastener embodiment in accordance with the invention, showing the fastener in different states of openness.

FIGS. 13 and 14 illustrate perspective views of another alternative surgical fastener embodiment in accordance with the invention, in an open and a closed configuration, respectively.

FIG. 15 is a cross sectional view of a person's stomach, depicting secured surgical fasteners coupled with a suture thread inside the stomach, such as would be performed in a gastric restrictive operation for morbid obesity.

FIG. 16 is partially a perspective view and partially longitudinal cross-sectional view of an alternative embodiment of an endoscopic fastening system in accordance with the invention.

FIG. 17 is a perspective view of a distal end of the embodiment of FIG. 16 depicting forceps jaws, and a cutaway cross section of a shaft member demonstrating a fastener magazine of the device in accordance with the invention.

FIGS. 18-22 are perspective views of the embodiment shown in FIGS. 16 and 17, showing successive steps in the fastening of a severed tubular structure, in accordance with the invention.

FIG. 23 is a partial perspective view, partially in cross section, of yet another embodiment of a fastening system in accordance with the invention.

FIGURE-24 is a perspective view of a distal collar of a fastener delivery and deployment assembly of the embodiment of FIG. 23.

FIGS. 25 and 26 are two-dimensional views of the fastener of a fastener used in the embodiment of FIGS. 23 and 24, showing the fastener in a closed and opened configuration in accordance with the present invention.

FIGS. 27 and 28 are perspective views of the fastener shown in FIGS. 25 and 26.

FIGS. 29-32 represent perspective views of several stages in the delivery and insertion of fasteners into target tissue using the embodiment of FIGS. 23-28, in accordance with the embodiment of the present invention.

For purposes of clarity and brevity, like elements and components will bear the same designations and numbering throughout the FIGURES.

### DETAILED DESCRIPTION

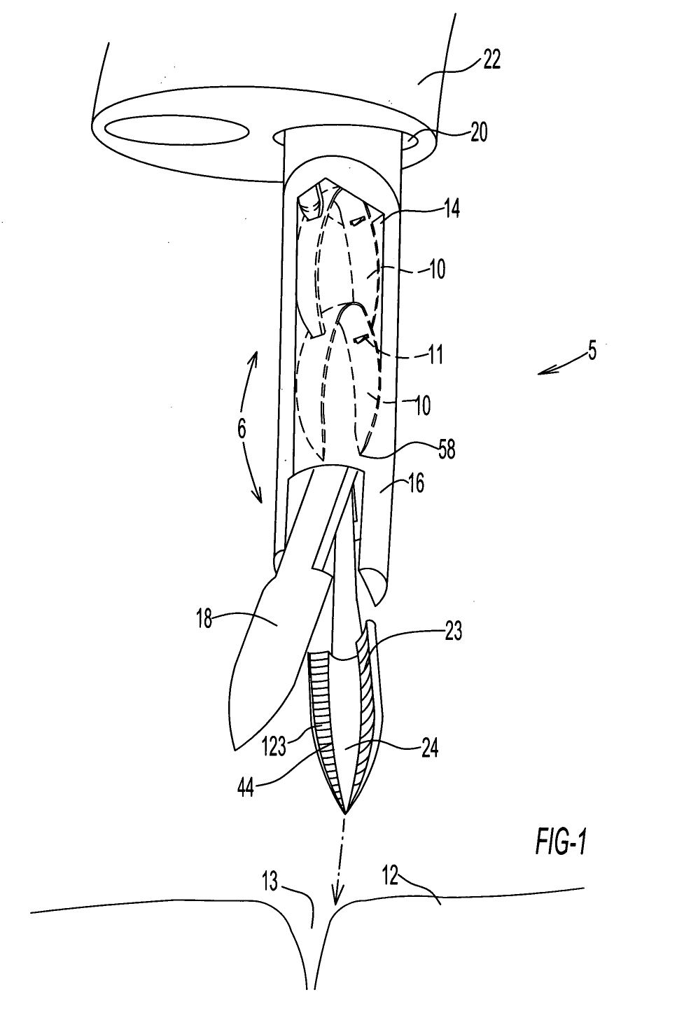

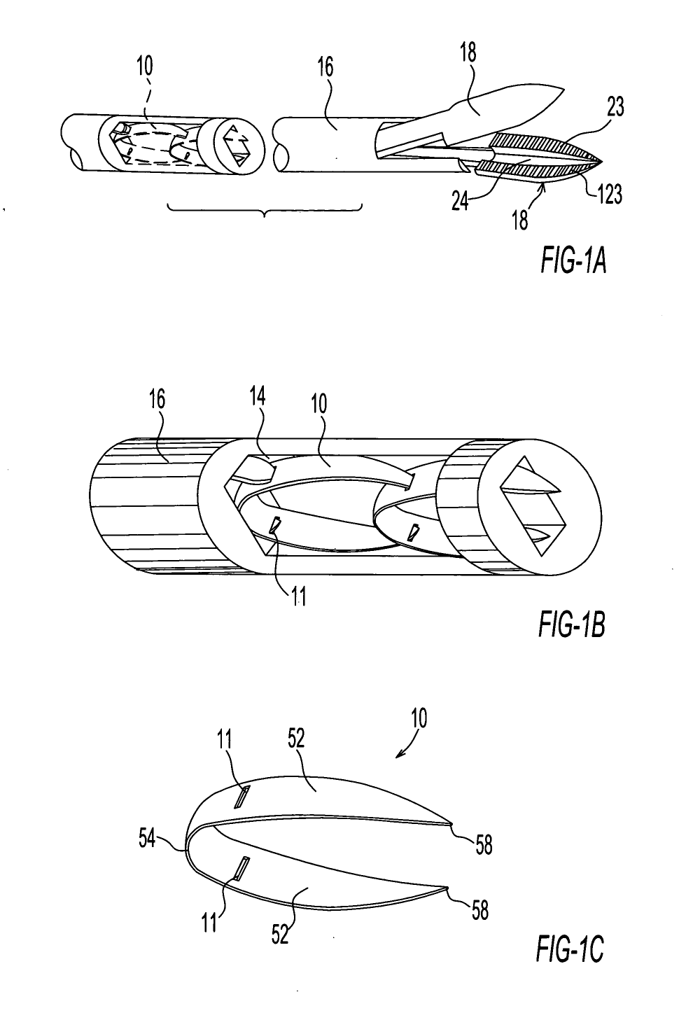

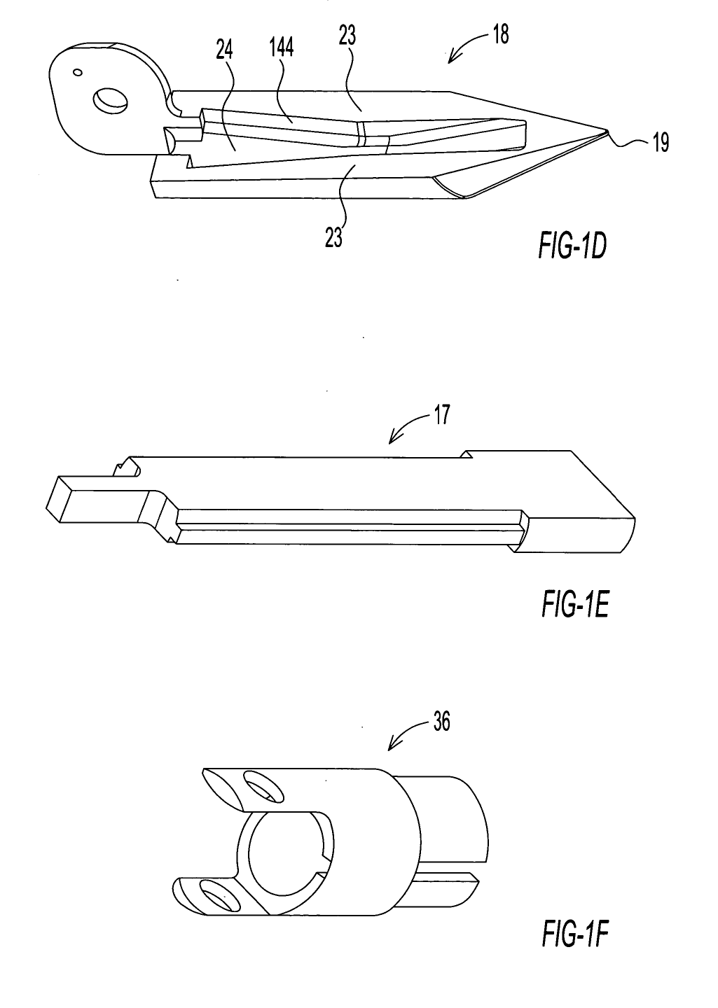

As illustrated in FIG. 1, an endoscopic fastening system 5 comprises one or more surgical fasteners 10 and a fastener delivery and deployment assembly 6, the assembly having a diameter sufficiently small so that it may be slidably insertable into an outer tubular member 20 in the form of a working channel extending longitudinally through an endoscope 22. The endoscopic fastening system, in one embodiment, for example, may be used in conjunction with an endoscope 22 having a working channel 20 2.8-4.2 mm in diameter and a shaft length of 230 cm.

Fastener delivery and deployment assembly 6 comprises a tubular shaft member 16, the shaft member being provided with a channel or lumen 14 extending longitudinally through the shaft member. Shaft member 16 is further provided at a distal end thereof with forceps jaws 18 configured for penetration of target tissue 12 and for closing fastener 10, as discussed further herein below. As shown in FIG. 1D, forceps jaws 18 are provided with preformed grooves 24 extending longitudinally on an inwardly facing surface of at least one of the jaws, for temporarily holding a fastener 10 therein. Forceps jaws 18 are formed with pointed tips 19 for piercing target tissue and with forceps jaw flats 23 for approximating and clamping the target tissue. Flats 23 may be formed with a plurality of transverse ridges and grooves 123 for facilitating the gripping of organic tissue during a fastening operation.

As shown in FIG. 1C, surgical fastener 10 comprises two legs or prongs 52 and a bight portion 54 joining the legs to one another. The distal ends of the fastener's legs 52 are provided with sharply pointed tips 58 to ensure piercing of target tissue for proper fastener entry. When stored inside shaft member 16 (FIGS. 1A and 1B), fastener 10 is disposed in a partially closed prefiring configuration inside channel or lumen 14, this channel being configured for maintaining the fastener in a partially closed prefiring configuration. In the partially closed prefiring configuration of fastener 10, legs or prongs 52 extend generally parallel to one another. During use of the fastening assembly 5 of FIG. 1, fasteners 10 never assume a configuration more open that that inside channel or lumen 14. The predetermined distance between legs or prongs 52 inside channel or lumen 14 is the maximum spacing that the legs or prongs have during use of the fastening system 5. Legs or prongs 52 may maintain their spacing by virtue of internal stresses of the material of which the fasteners 10 are made. A plurality of second fasteners disposed end to end forming a fastener-magazine are contained within channel or lumen 14, the fastener magazine enabling successive fastening applications without necessitating removal of the device 5 from the endoscope 22 each time a fastener 10 is applied.

In a particular embodiment of the invention, namely, endoscopic fastening system 5 depicted in FIGS. 1-6, fastener 10 is disposed in its partially closed prefiring configuration as it travels through channel or lumen 14.

FIG. 1 shows endoscopic fastening system 5 disposed within the distal end of endoscope 22 during an endoscopic or laparoscopic procedure wherein forceps jaws 18 are shown in the open position and positioned over target tissue 12. Specifically, the jaws are positioned over a surgical site 13 or other portion of tissue 12 that is to be closed with one or more fasteners 10.

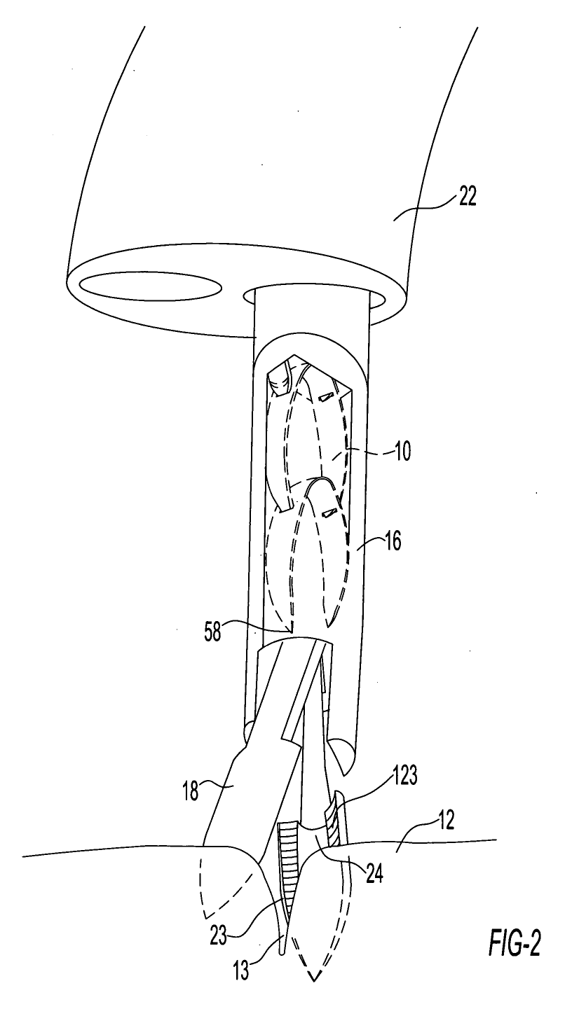

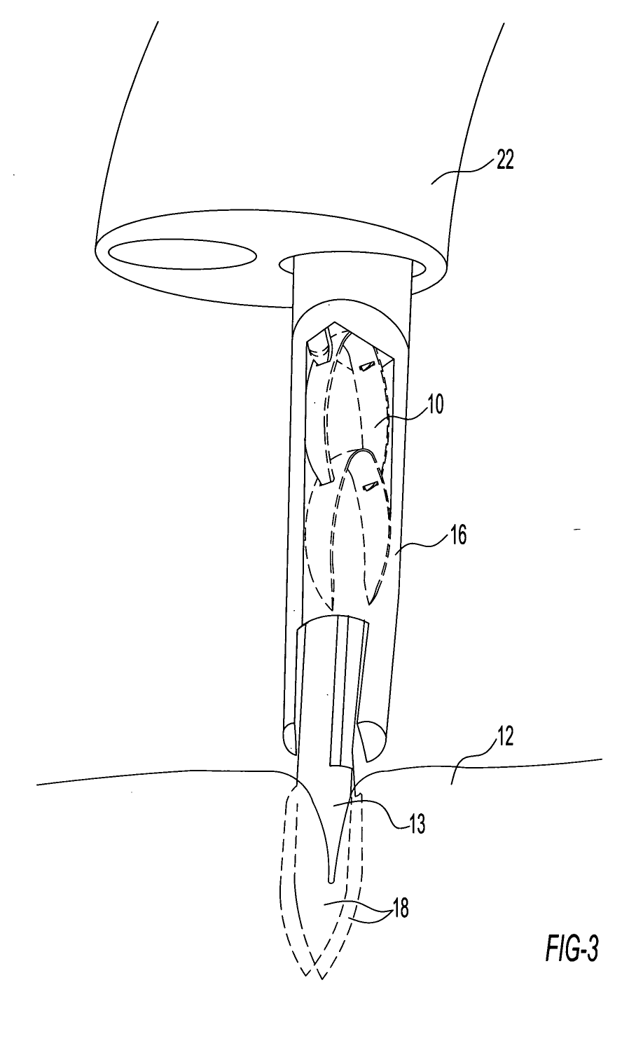

Referring to FIG. 2, shaft member 16 is advanced from endoscope working channel 20 to exert a force on sharply pointed forceps jaws 18 causing the forceps jaws to penetrate the target tissue 12 proximate operative site 13 in an open configuration with all fasteners 10 still securely proximal to the jaws. When fully inside target tissue 12, forceps jaws 18 close upon incision 13, approximating both walls of the incision by means of clamping flats 23, as shown in FIG. 3.

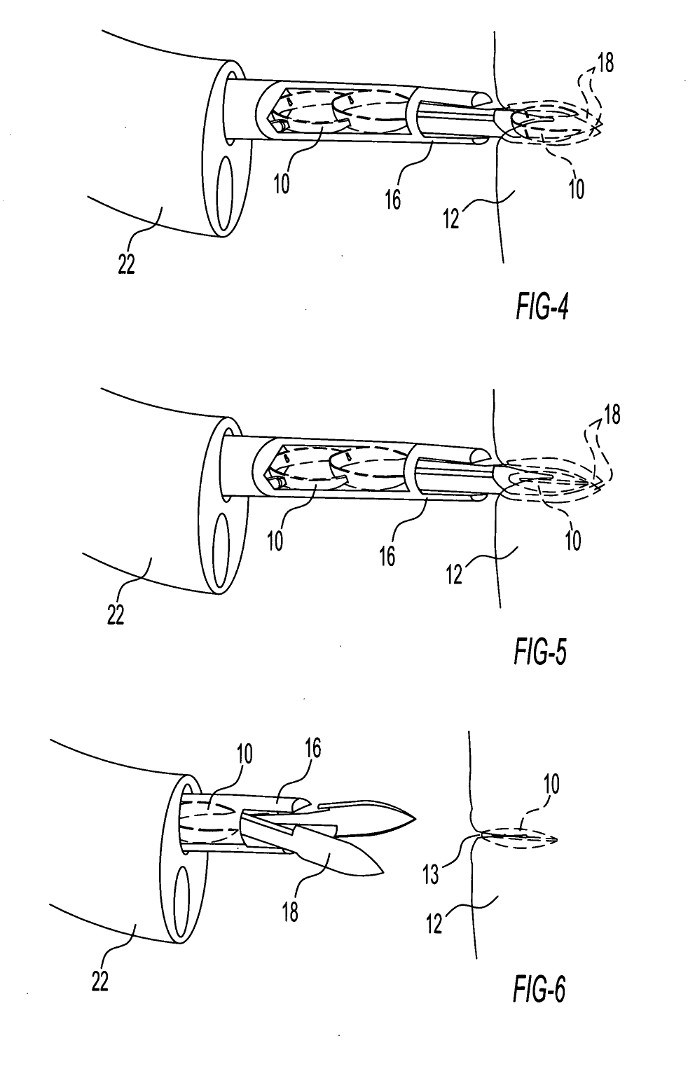

Once proper placement of forceps jaws 18 upon incision 13 is accomplished, fastener 10 is advanced in a distal direction into preformed grooves 24 of forceps jaws 18, its legs 52 entering target tissue 12 (FIG. 4). Forceps jaws 18 are then closed further upon fastener 10, thereby permanently deforming the fastener's bite portion 54, causing fastener 10 to assume its fully closed postfiring configuration (FIG. 5), whereupon the forceps jaws are opened and extracted from the target tissue 12, thereby releasing the fastener 10 therein (FIG. 6).

The particular embodiment depicted in FIGS. 1-6 represents an important innovation in the technology of small fasteners (staples, clips, etc). This embodiment precludes the need for the “opened-closed-opened” method, i.e. necessitating a fastener to dwell inside an inner tubular member in a closed configuration, be opened by some means when deployed before entering a target tissue, and then closed again once inside the target tissue. The particular embodiment of this invention provides for a “closed-closed-closed” method, thereby simplifying the design of a fastening system to be used in conjunction with an endoscope. Pursuant to the embodiment of FIGS. 1-6, fastener or staple 10 is disposed inside channel 14 with legs 52 in a substantially parallel configuration that is geometrically close to the closed use configuration, after crimping of bight, 54 by forceps jaws 18. Fastener 10 never has a configuration which is more open that the pre-firing configuration inside channel 14 of tubular shaft member 16. Fastener 10 only enters the target tissue 12 after the forceps jaws 18 have approximately the tissue. During the subsequent locking of fastener 10 inside the target tissue 12, the deformation of bight 54 is accomplished with little change in the relative positions of legs 10.

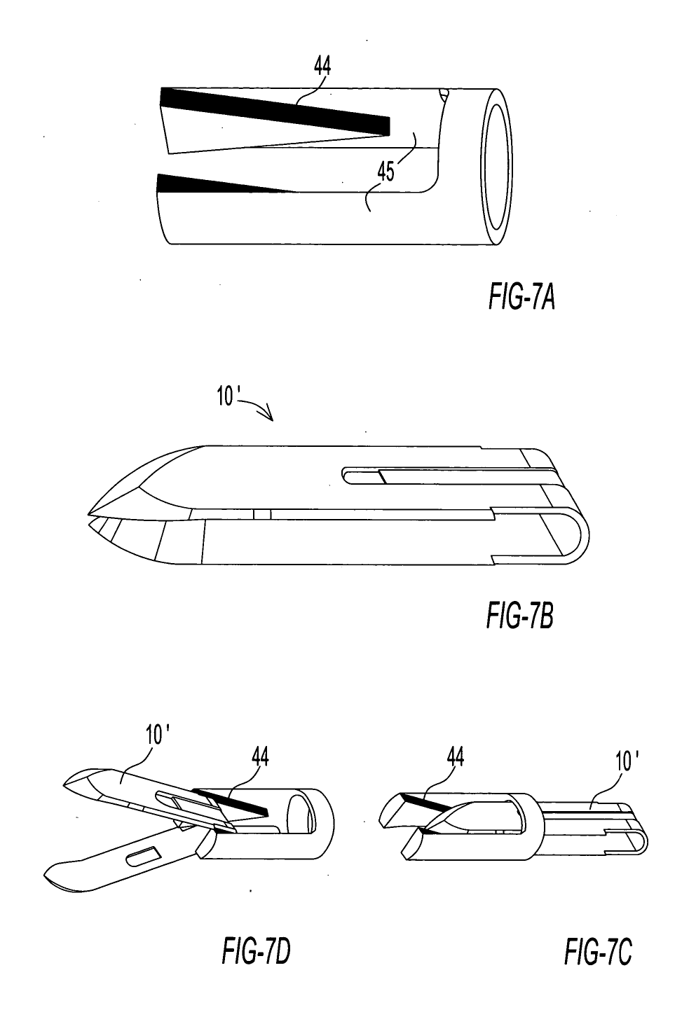

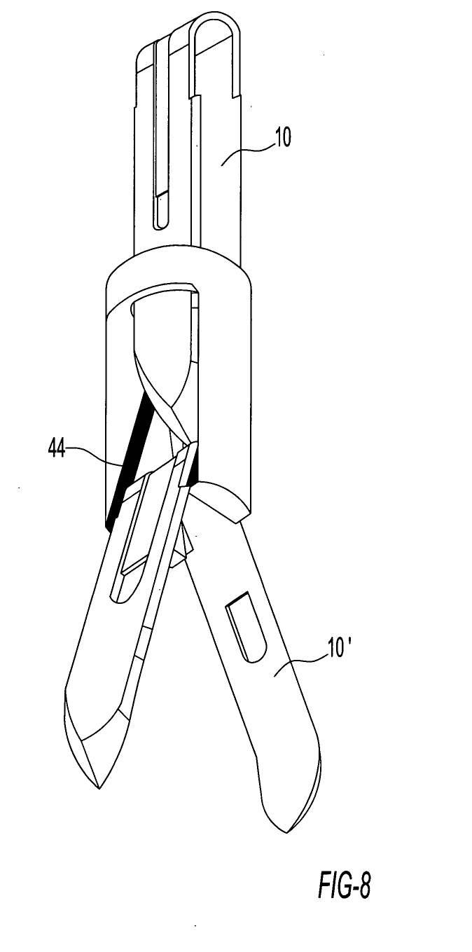

As depicted in FIGS. 7A-7D and 8, a somewhat different embodiment utilizes shaft member 16, forceps jaws 18, an actuation assembly, and one or more fasteners 10′ housed inside channel 20 as described in the embodiment of FIG. 1-6. However, as illustrated in FIGS. 7A-D, and FIG. 8, the method of fastener advancement, preparation before firing, and deployment is that of the “closed open closed” method.

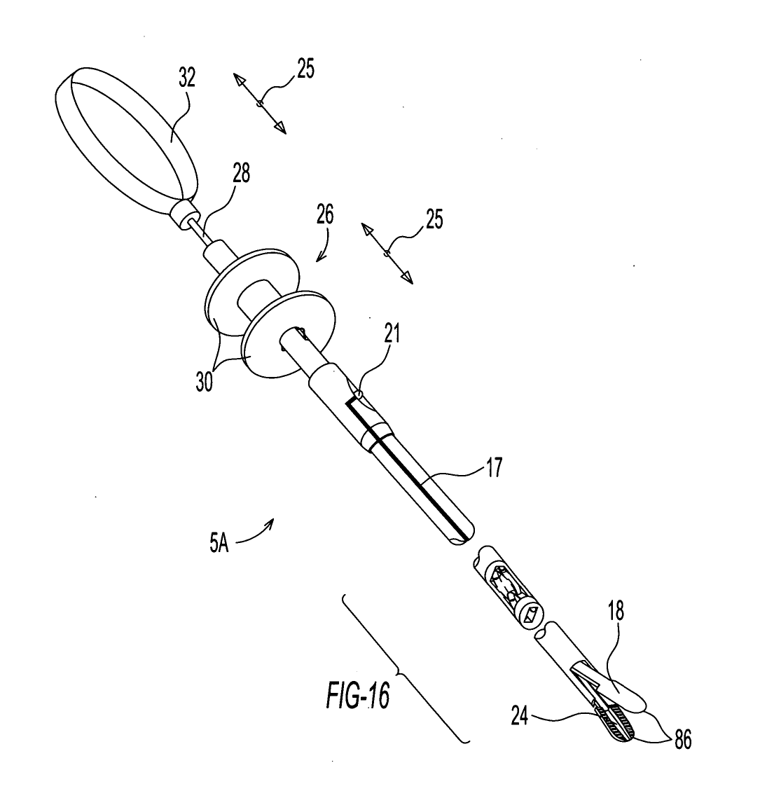

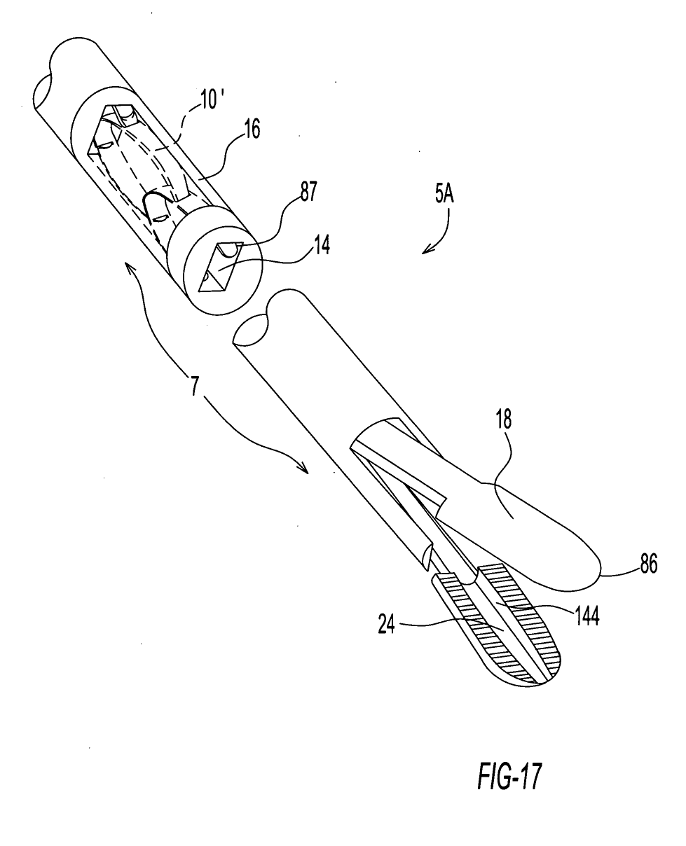

In a particular endoscopic fastening system 5A (FIGS. 16 and 17), a clevis subassembly 36 is provided (FIG. 1F), the clevis subassembly being proximally disposed to forceps jaws 18, is provided with at least one forming rail 44 (FIG. 7A), the forming rail being operatively coupled proximally with tubular shaft member 16, and distally, with forceps jaws 18. A pair of triangular or wedge-shaped rail members 44 are disposed along each arm or leg 45 of a clevis portion 36 of tubular shaft member 16. When a fastener 10′ travels in a distal direction from channel or lumen 14 to forceps jaws 18, the fastener member 10′ traverses the clevis subassembly 36. Referring to FIGS. 7C and 7D respectively, fastener 10′ passes along forming rail 44, and is thereby forced from a partially closed prefiring configuration into an open prefiring configuration. FIG. 8 illustrates two such fasteners 10′ with a more proximally located fastener being in the partially closed prefiring configuration on its way into the forming rails 44, while a more distally located fastener has already traversed the forming rails 44, and is disposed in an open prefiring configuration.

A pusher rod member 17 (FIGS. 1E and 16) slidably disposed inside channel or lumen 14 of shaft member 16 is provided for advancing a line or magazine of fasteners 10′ in a distal direction towards forceps jaws 18 upon firing of a first fastener 10′, i.e., upon placement of that first or most distal fastener inside target tissue. Jaws 18 are fed fasteners 10′, generally one at a time, the jaws being actuated to place and secure the fasteners as described herein below.

Referring to FIG. 16, endoscopic fastening system 5A includes a fastener delivery and deployment assembly 7 with an actuation mechanism 26 used for manipulating jaws 18, and loading and positioning fasteners 10 or 10′, the mechanism being used generically in all embodiments disclosed herein. Actuation mechanism 26 comprises one or more drive wires 28, the drive wires being operably configured to actuate forceps jaws 18. Various different configurations may be employed to impart motion to forceps jaws 18 through the manipulation of the actuation mechanism 26, as will be understood by those skilled in the art.

Actuation mechanism 26 includes a finger spool 30 and a thumb ring 32 maneuverable with respect to one another in order to actuate jaws 18. Comparable to a biopsy forceps, jaws 18 are opened and closed by means of manipulating actuation mechanism 26, the mechanism being proximate the proximal end of a fastener delivery and deployment assembly. Finger spool 30 and thumb ring 32 may-be moved closer together or farther apart, as shown by arrows 25, in order to move drive wires 28 and actuate jaws 18.

Actuation mechanism 26 also includes a slide subassembly 21, the subassembly being configured for sliding along shaft member 16 (FIG. 16). Slide subassembly 21 is operatively coupled with pusher rod member 17 such that when slide subassembly 21 and pusher rod member 17 are moved in a distal direction, the pusher rod member pushes fasteners 10 or 10′ along channel or lumen 14. Slide subassembly 21 may be provided with a series of marked positive stops or controlled slide distances (not shown) that operatively correlate with the distance that each single fastener 10 or 10′ will travel into jaws 18. Accordingly, only one fastener 10 or 10′ may be moved into forceps jaws 18 at a time.

When fasteners 10 or 10′ are loaded, pusher rod member 17 is in a retracted position and approximates contact with the proximal end of the most proximal fastener. Pusher rod member 17 is configured and used to advance fasteners contained in channel or lumen 14 along shaft member 16, one fastener at a time.

Returning to FIG. 17 and endoscopic fastening system 5A, fastener 10′ may be made of surgical stainless steel and preferably formed of a metal capable of being bent into acute radii of a partially closed prefiring configuration without developing significant structural weakness. As such, fasteners 10′ may pass through fastener delivery and deployment assembly 7 and channel or lumen 14 of elongate shaft member 16. Fasteners 10′ may pass through channel 14 of tubular shaft member 16 in a partially closed prefiring configuration, be forced into an open prefiring configuration when traversing forming rail 44 of clevis 36, and be fully closed to their post firing configuration inside the target tissue as described further herein below.

Referring again to FIG. 1C, fastener 10 is configured such that its legs 52 are each provided with a transversely oriented fastener groove 11 formed on an outer surface. Tips 58 of each fastener 10 are configured for engaging or inserting in fastener grooves 11 of the respective distally adjacent fastener such that fasteners 10 may nestle together inside channel or lumen 14. This linear engagement of the fasteners 10 is designed to allow a more proximally positioned fastener to drive a more distal, the most proximal fastener being pushed by pusher rod member 17. Grooves 11 of fasteners 10 are used in conjunction with preformed grooves 24 in forceps jaws 18 for directing fasteners 10 into a proper position within the forceps jaws 18.

In endoscopic fastening systems 5 and 5A, a magazine of staples described hereinabove might be contained in channel or lumen 14 of shaft member 16 as illustrated in FIGS. 1-6 and 16-17. Forceps jaws 18 are operatively coupled with actuation mechanism 26 (FIG. 16), the actuation mechanism including drive wire(s) 28 and pusher rod member 17. Shaft member 16 contains pusher rod member 17, the pusher rod member sliding distally to advance fasteners 10, and also contains drive wire(s) 28 utilized for opening and closing forceps jaws 18. Slide mechanism 21 of actuation mechanism 26 is operatively connected to usher rod member 17 for controlling the forward motion thereof (FIG. 16).

Referring to FIG. 17, a perspective view of shaft member 16 and channel or lumen 14 is shown, wherein preformed groove 24 is configured for guiding and containing fastener 10 as fastener 10 is advanced from channel 14 along a pair of guide rails 144 into open forceps jaws 18 by actuation of pusher rod member 17. Preformed groove 24 is provided with opposing rails 144, the rails exemplarily taking the form of longitudinal walls of groove 24 whereby froove 24 functions as a fastener guide. Walls or rails 144 may be tapered from a proximal end to a distal end of groove 24, for facilitating the reception and realignment of fasteners 10 (or 10′).

With further reference to FIG. 17, the distal ends of fasteners 10 engage rails 144 on either side of preformed groove 24, thereby orienting fastener legs 52 into preformed grooves 24 of jaws 18. As such, when a fastener 10 is positioned in jaws 18, the fastener is opposing surgical site 13 ready for forceps jaw closure upon target tissue. For certain types of operations entailing tissue approximation and proper penetration, jaws 18 and fastener 10 are configured to be long and slender (see, e.g., FIGS. 1-6). In addition, forceps tips 19 as well as fastener tips 58 are sharpened, pointed and/or otherwise configured to penetrate into target tissue 12. On the other hand, and as illustrated in FIGS. 16 and 17, forceps jaws 18, as well as the fasteners, may be provided with rounded tips for other operations to be described herein below.

Within reference to FIG. 1D, forceps jaws 18 are also provided with laterally positioned flat clamping surfaces (“flats”) for clamping and approximating target tissue 12 prior to fastening. Flat clamping surfaces 23 may be grooved or ridged for better tissue gripping, or smooth for better tissue penetration as is required.

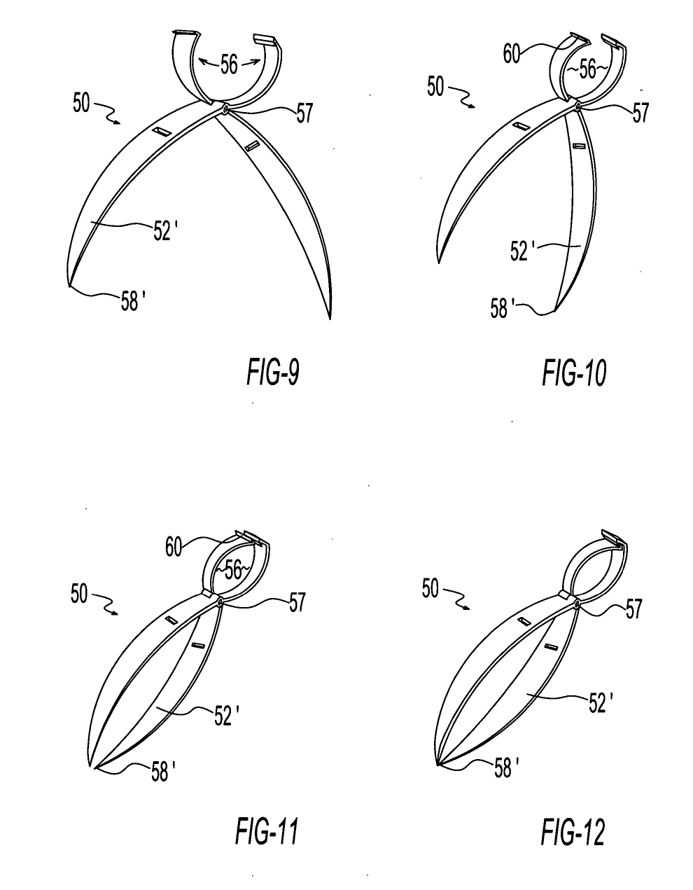

FIGS. 9-12 illustrate another fastener 50 comprising two legs 52′ co-joined by a hinge mechanism 57 proximate the proximal end of fastener 50. The two legs 52′ have pointed distal ends similar to fastener 10 and move relative to one another in a scissors-like fashion. A locking mechanism 56 comprising a pair of leg extensions is disposed proximal to hinge mechanism 57. When fastener 50 and more particularly locking mechanism 56 is squeezed by the forceps jaws 18, the fastener 50 assumes its fully closed postfiring configuration, with hook-shaped clasping formations 60 at the proximal ends of leg extensions 56 interlocking to hold fastener 50 closed. FIGS. 11 and 12 illustrate fastener 50 being closed with locking mechanism 56 having been activated.

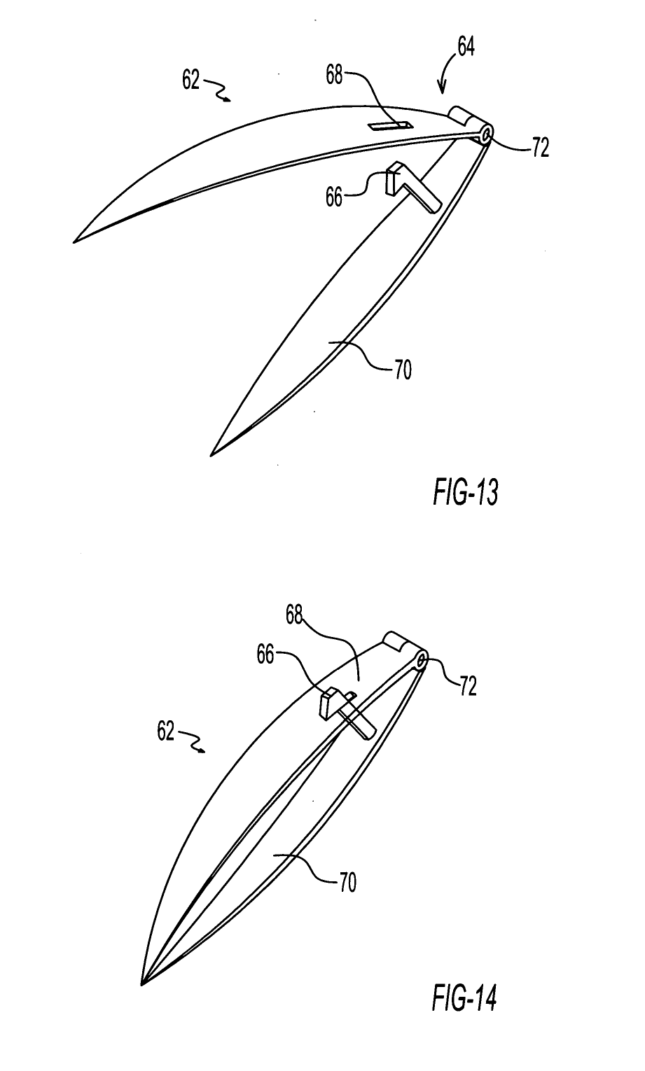

FIGS. 13 and 14 illustrate an alternative fastener 62. The fastener is configured with a locking mechanism 64, the mechanism being provided with a hook structure 66, the hook structure fitting into an appropriately formed slot 68. Two legs 70 of fastener 62 are coupled together by a hinge structure 72. When the fastener is deployed, the legs 70 are brought together, and hook structure 66 engages slot 68 as illustrated in FIG. 12. Hook structure 66 locks legs 70 together such that fastener 62 is locked into position, as on a target tissue. Hinge structure 72 optionally incorporates spring loading.

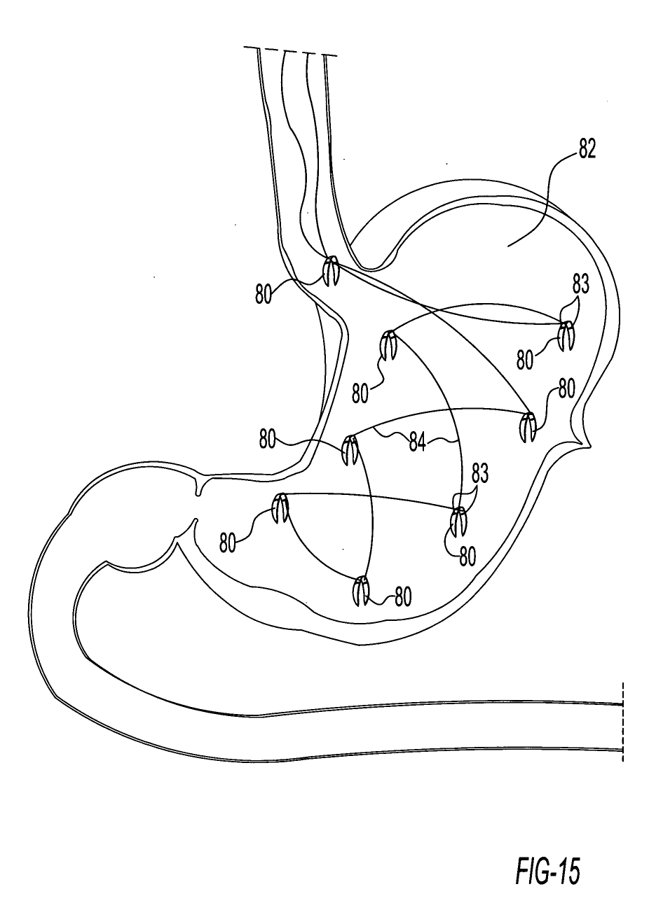

As illustrated in FIG. 15, a plurality of surgical fasteners 80 are provided with cavities 83 proximate the fasteners' proximal end, the fasteners being Coupled one to another by suture thread 84. Fasteners 80 may be applied in a crisscross fashion into two opposing walls of a stomach 82 during gastric restrictive surgery. When such a fastener application is completed, it simulates an open shoelace. The two loose ends of suture thread 84 are then cinched together, causing opposing stomach walls to approximate, thus achieving restriction of the gastric pouch or stomach 82.

Alternatively, fasteners 80 may be applied along opposite edges of a tissue cut with suture sections spanning across the cut. Pulling the sutures may then cinch together the tissue to provide desired tissue closure. FIGS. 16 and 17 illustrate an alternative endoscopic fastening system 5A, whereby forceps-tips 86 and fastener-tips 87 are rounded (see FIG. 17). The fasteners used in this embodiment may be utilized for clamping free-standing tissue, rather than penetrating or entering through its wall. For example, a severed blood vessel, or other tubular body structure such as a bile duct may be clamped with the fastener.

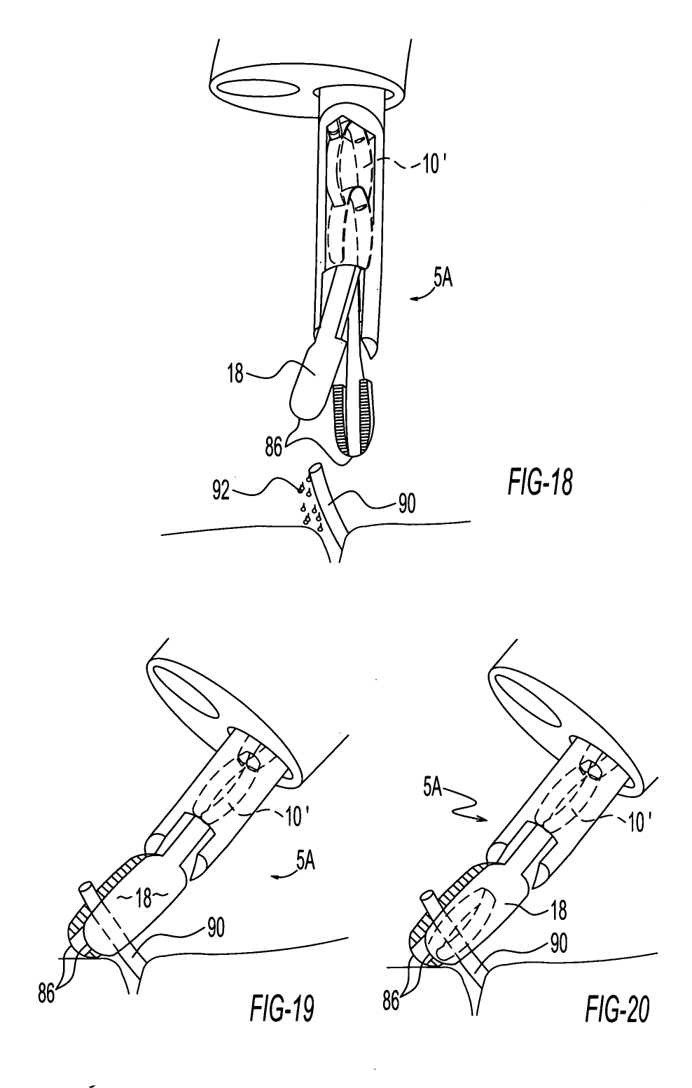

Referring to FIG. 18, a target tissue may consist of a severed tubular structure 90, the structure having been cut or otherwise lacerated such that it leaks blood or some other fluid 92. To close severed tubular structure 90, forceps jaws 18 with blunt or rounded tips 86 are positioned proximate severed tubular structure 90, the structure being clamped with forceps jaws 18 in order to close the structure and arrest the flow of fluid (FIG. 19). As shown in FIG. 20, clamped tubular structure 90 is compressed such that a partially closed fastener 10 with blunt or rounded distal tips 87 may slide over the compressed tubular structure without having to further open the fastener. Next, forceps jaws 18 are closed further, causing fastener to assume its fully closed postfiring configuration, thereby clamping tubular structure 90 shut (FIG. 21). Once a fastener has been applied, forceps jaws 18 may be opened and the fastening system withdrawn as illustrated in FIG. 22.

Yet another embodiment of a fastener application assembly is described herein below, the embodiment constituting an advance over the disclosed subject matter of U.S. Provisional Application No. 60/557,201, filed on Mar. 29, 2004, and Provisional Application No. 60/611,257, filed on filed Sep. 17, 2004. In the following discussion, like reference numerals are used to designate like elements, relative to embodiments described above.

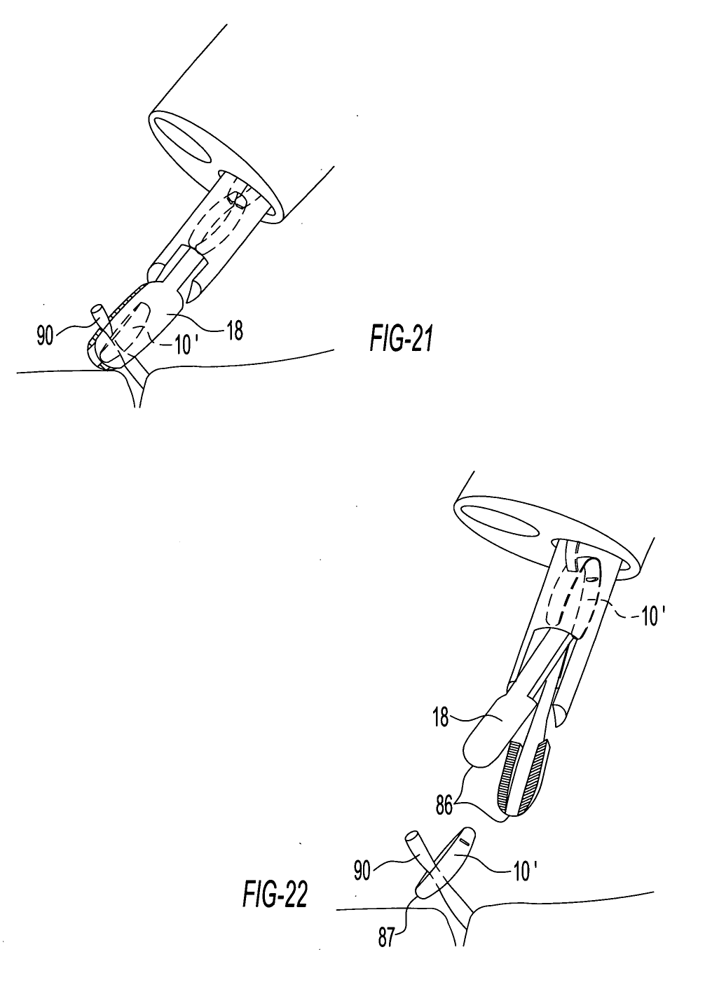

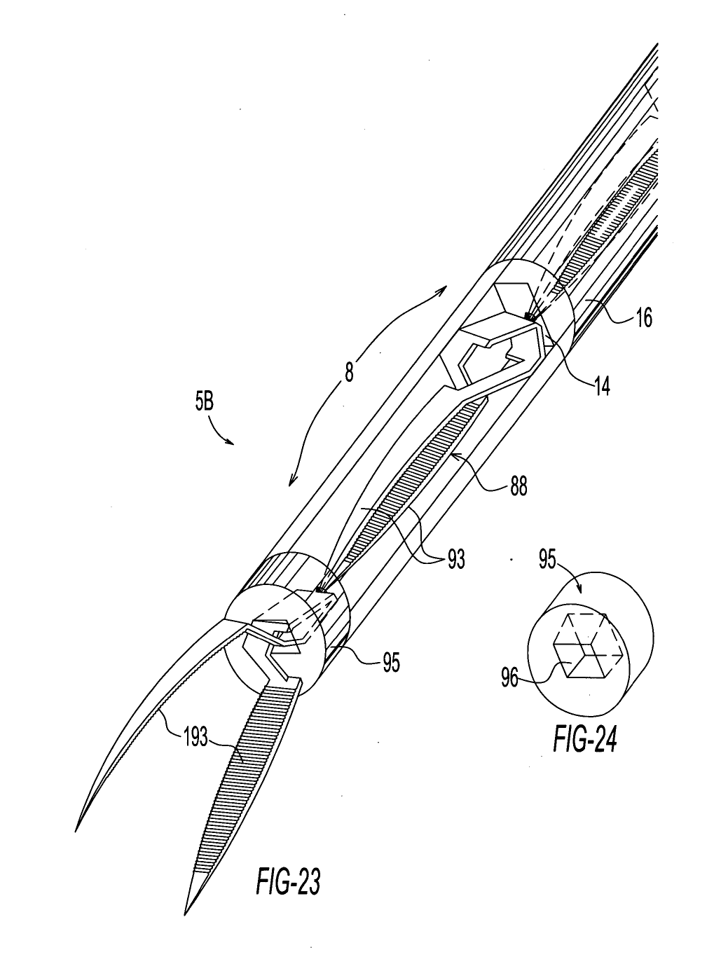

An endoscopic fastening system 5B provided with a fastener delivery and deployment assembly 8 shown in FIG. 23 includes a shaft member 16, an channel or lumen 14, and fasteners 88 disposed in a magazine fashion within channel or lumen 14.

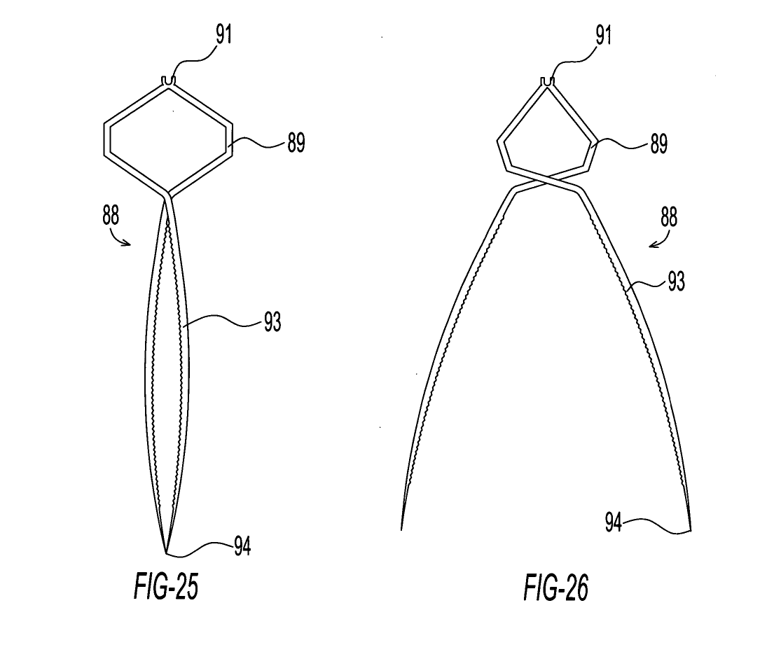

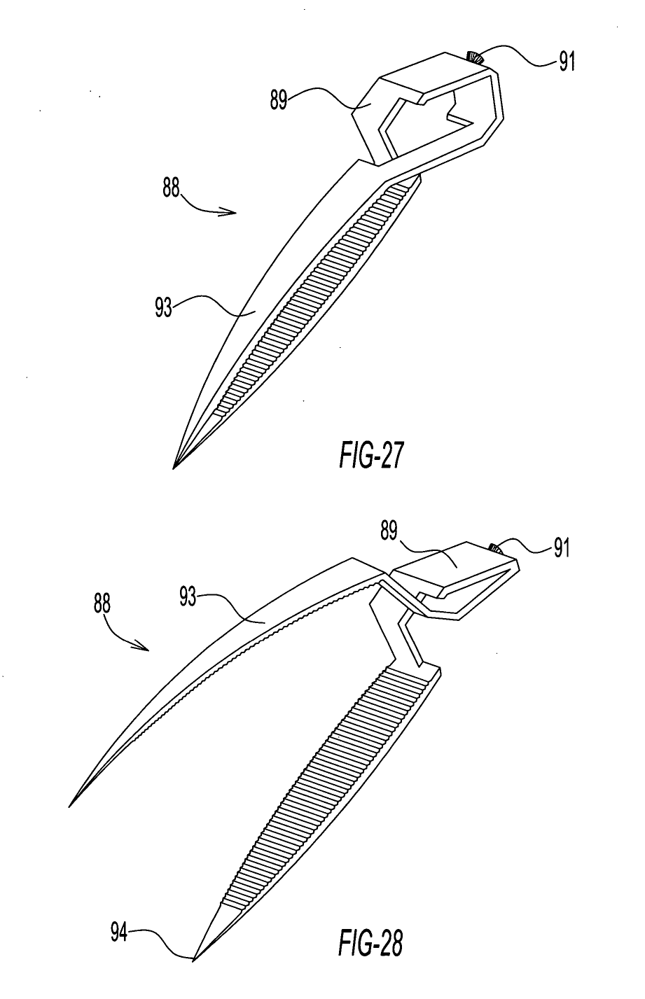

In FIGS. 25 and 27, fastener 88 is depicted in a closed configuration, the fastener being made from a spring biased material such as plastic or certain spring biased surgical stainless steel, or a shape memory material such as Nitinol (NiTi). Fastener 88 is provided with two slender legs or prongs 93, the prongs configured with pointed distal ends and move relative to one another in a scissors-like fashion. A spring member 89 (FIGS. 25-28) operatively connected to prongs 93 is provided on a proximal aspect of fastener 88. At a trailing end of spring member 89 is provided a cup or recess formation 91 for receiving the tip of a following fastener in a staple magazine.

In FIGS. 25 and 27, fastener 88 is depicted in an unstrained closed configuration. When pressure is applied onto outer lateral surfaces (not separately designated) of spring member 89, prongs 93 of fastener 88 are forced into an open configuration. When the pressure is released, prongs 93 spring back into a closed configuration. Fastener 88 is disposed in a closed prefiring configuration as it traverses channel or lumen 14 (FIG. 23).

Referring now to FIGS. 23 and 24, shaft member 16 is provided with a collar member 95 made of hard material such as, for example, stainless steel, the collar member being configured such that the diameter of its inner lumen is smaller than that of channel or lumen 14, providing a restrictive lumen or passageway 96for purposes of exerting a transverse force on spring member 89 to open fastener 88 as the fastener and particularly the spring member portion thereof traverses lumen or passageway 96 during a distal motion of the fastener magazine or stack. Restrictive lumen 96 may be tapered down, that is, shaped with a wider proximal opening contiguous with channel or lumen 14, the proximal opening of restrictive lumen or passageway 96 being configured for guiding the distal ends of fastener 88 into collar member 95. Restrictive lumen 96 thus has the shape of a truncated pyramid.

Prongs 93 may be provided with preferably transversely oriented ridges 193 along at least one inner surface of the prongs, the ridges being provided for grasping target tissue. Prongs 93 may alternatively be configured with smooth inwardly facing surfaces, without ridges, for easy entry into target tissue. As shown in FIGS. 25-28, prongs 93 are also provided with sharply pointed prong-tips 94 for easy entry into target tissue. Alternatively, prongs 93 of the fastener 88 may be provided with blunt or rounded tips, the fastener being designed for fastening tissue without tissue entry, such as blood vessels, ducts or other tubular structures.

Referring again to FIG. 23, fasteners 88 are disposed end to end to form a fastener magazine, contained inside channel or lumen 14 as described herein above. Fasteners 88 housed proximal to collar 95 are shown in an unstrained, closed configuration. A pusher rod member 17 not shown in FIG. 23, but illustrated in FIG. 16, is provided. The pusher rod member 17, being slidably disposed inside channel or lumen 14 of shaft member 16 is provided for advancing the entire magazine or stack of fasteners 88 in a distal direction towards collar member 95 upon firing of a first or most distal fastener 88 as described herein below.

When fasteners 88 are loaded, pusher rod member 17 is in a retracted position and approximates contact with the proximal end of the most proximal fastener. Pusher rod member 17 is configured and used to advance fasteners 88 contained in channel or lumen 14 along shaft member 16, one fastener at a time. When a most distal fastener's prongs 93 enter into collar 95, the prongs being in a closed configuration, prongs 93 pass through restrictive lumen 96 without obstruction, because the inner diameter of the restrictive lumen 96 is appropriately sized for such passage.

Spring member 89 of fastener 88 is larger than the inner diameter of restrictive lumen 96, so that the spring member becomes constricted or compressed during passage therethrough. Spring member 89 is guided into restrictive lumen 96 by traversing a graded narrowing or tapering of restrictive lumen 96. Thus, at the point of that restrictive lumen 96 is contiguous with a distal aspect of channel or lumen 14, the restrictive member's inner diameter equals that of channel or lumen 14. This diameter narrows progressively in a distal direction to the distal end of collar member 95. This most narrow part of restrictive lumen 96, at the distal end of collar member 95, is configured with a smaller diameter than the largest horizontal cross section of spring member 89, thus causing spring member 89 to constrict upon passing through restrictive lumen or passageway 96. The force exerted upon spring member 89 provides for the opening of prongs 93 into a pre-firing state as discussed herein above.

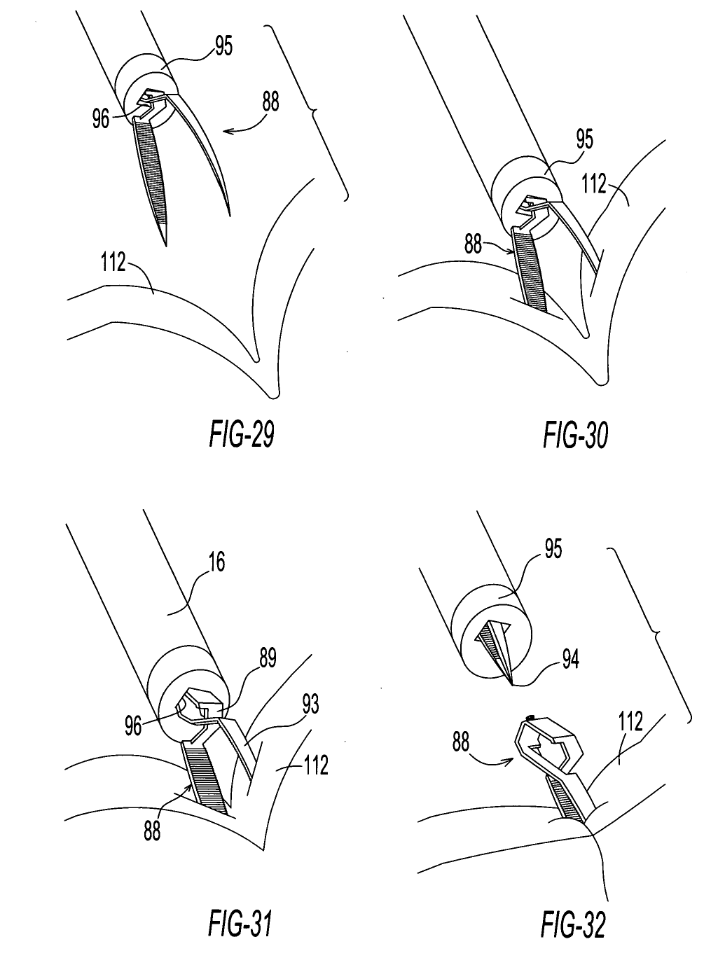

FIGS. 29-32 show the endoscopic fastening system 5B as incorporating an opened fastener 88 disposed inside restrictive lumen 96 of collar member 95, the fastener approaching a target tissue 112, the fastener being in a strained state owing to compression of spring member 89 by restrictive lumen 96. FIGS. 30 and 31 illustrate the entry of fastener 88 into target tissue 112. Upon a further push by a pusher rod member 17 (FIG. 16), spring member 89 of fastener 88 is released from restrictive lumen 96 (shown in FIG. 24), thereby releasing the tension on the spring member, and causing prongs 93 to snap closed

The above described embodiment may be configured with forceps jaws 18, the jaws having been described with respect to other embodiments of a fastener delivery and deployment assembly in connection with FIGS. 1-6 and 16-22.

In an embodiment configured with forceps jaws 18, the jaws would be used for clamping tissue as-described above. In a surgical procedure using forceps jaws 18, upon approaching a target tissue 112, the 18 jaws would be opened by means of an actuation mechanism as discussed above with reference to FIG. 16. Fastener 88 would then be advanced, and enter the opened jaws, the fastener being forced into an open configuration. Both forceps jaws and fastener would now be open and ready to be inserted into a target tissue.

Forceps jaws may be helpful in a case wherein a fastener would be too delicate or flimsy for providing proper tissue clamping, such as, for example, during a severe arterial bleed, or a large incision in need of approximation. On the other hand, if a smaller lesion requires closing, this embodiment may function well configured without forceps jaws, or a complex actuation mechanism, thereby providing cost savings, and simplifying design for manufacture of the device.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

1. An endoscopic fastening system for securing tissue internal to the body utilizing an endoscope, comprising:
an elongate fastener delivery and deployment assembly being provided at a distal end with forceps jaws and at the proximal end with an actuation mechanism for manipulating said jaws between an open and a closed position;
one or more surgical fasteners disposed inside said elongate fastener delivery and deployment assembly, said fasteners having a predetermined position relative to said jaws, said fastener being disposed in a substantially closed pre-firing configuration in said assembly proximally of said jaws prior to an opening of said jaws, said fastener having legs or prongs that are disposed at a predetermined distance from one another in said closed pre-firing configuration; and
a pusher rod member provided for advancing said fasteners in a distally directed motion of said pusher rod member,
at least one of said elongate fastener delivery and deployment assembly and said fastener having means for constraining said legs or prongs to have a spacing at all times at most as great as said predetermined distance, whereby said fastener may be moved in a substantially closed configuration into said jaws and from said jaws into target tissue.

2. The endoscopic fastening system in accordance with claim 1 wherein said fastener is constrained by internal stresses to maintain said predetermined distance between said legs or prongs, said fastener being deformable so that said legs or prongs may be brought into a fully closed postfiring configuration wherein said legs or prongs are spaced from one another by a distance smaller than said predetermined distance.

3. The endoscopic fastening system in accordance with claim 2, wherein a fastener bight portion is permanently deformable to produce said fully closed postfiring configuration.

4. The endoscopic fastening system in accordance with claim 1, wherein said assembly has an outer diameter sufficiently small to enable insertion of said assembly through a working channel of a flexible or rigid endoscope.

5. The endoscopic fastening system in accordance with claim 1 wherein said forceps jaws are configured for engaging a fastener when said jaws are in a partially closed configuration.

6. The endoscopic fastening system in accordance with claim 5, wherein said forceps jaws are provided with preformed grooves extending longitudinally on a medial aspect of an inwardly facing surface of at least one of said jaws.

7. The endoscopic fastening system in accordance with claim 1 wherein said forceps jaws are configured for approximating and/or clamping severed target tissue or other disparate segments of tissue.

8. The endoscopic fastening system in accordance with claim 7, wherein said forceps jaws are provided with flat surfaces extending longitudinally on a lateral aspect of an inwardly facing side of at least one of said jaws.

9. The endoscopic fastening system in accordance with claim 8, wherein said surfaces are provided with ridges configured for tissue approximation and/or clamping.

10. The endoscopic fastening system in accordance with claim 1, wherein said forceps jaws are provided with pointed tips, said tips being provided for tissue penetration.

11. The endoscopic fastening system in accordance with claim 1, wherein said legs or prongs are provided with pointed distal tips for facilitating tissue penetration.

12. The endoscopic fastening system in accordance with claim 1, wherein said fastener is provided with a closure means for maintaining said fastener in a fully closed post-firing configuration.

13. The endoscopic fastening system in accordance with claim 12, whereby said closure mechanism comprises a hook and slot mechanism.

14. The endoscopic fastening system in accordance with claim 12, whereby said closure mechanism comprises of a hinge mechanism.

15. The endoscopic fastening system in accordance with claim 12, whereby said closure mechanism is comprised of a spring mechanism.

16. The endoscopic fastening system in accordance with claim 1, wherein said fastener is one of a plurality of identical fasteners disposed in a partially closed prefiring configuration within a channel of a tubular member, said tubular member maintaining said fasteners in said partially closed prefiring configuration to form a fastener-magazine within said inner tubular member.

17. The endoscopic fastening system in accordance with claim 1, wherein said actuation mechanism is provided with a slide subassembly, said subassembly being configured for sliding along a shaft member.

18. The endoscopic fastening system in accordance with claim 17, further comprising a pusher rod member operatively connected to said slide subassembly so that sliding of said slide subassembly induces said pusher rod member to push a magazine of fasteners along said shaft member in a distal direction.

19. The endoscopic fastening system in accordance with claim 18, whereby said slide subassembly is provided with means to control each fastener's separate delivery into said forceps jaws.

20. An endoscopic fastening system for securing tissue internal to the body, comprising:
an elongate fastener delivery and deployment assembly provided at a distal end with forceps jaws and at a proximal end with an actuation mechanism; and
a surgical fastener, said fastener comprising two legs and a bight portion joining said legs to one another, said fastener being disposed inside said elongate fastener delivery and deployment assembly, said fastener delivery and deployment assembly including an elongate shaft member provided with a channel extending longitudinally therethrough,
said channel housing one or more fasteners disposed in a magazine;
said fastener delivery and deployment assembly further including a pusher rod member slidably disposed inside said channel, said pusher rod member being provided for advancing fasteners in a distal direction along said shaft member;
said fastener delivery and deployment assembly further including forceps jaws provided with preformed grooves, and flat surfaces, said jaws being configured for engaging each fastener and approximating tissue respectively, said jaws being provided with pointed tips for easy tissue penetration; and
said actuation mechanism being provided for for at least one action taken from the group consisting of fastener positioning inside said forceps jaws, and effectuating said jaws alternately into opened and closed positions.

21. A method for performing surgical operations on internal body tissues of a patient, comprising:
(i) shifting a fastener delivery and deployment assembly in a distal direction towards target tissue, said assembly including forceps jaws disposed in an initial closed configuration at a distal end;
(ii) operating an actuator on said assembly to open said forceps jaws (iii) plunging the opened jaws into said target tissue;
(iv) after the plunging of the opened jaws into said target tissue, closing said forceps jaws to a first closed configuration, thereby causing tissue approximation;
(v) after the closing of said forceps jaws, advancing a first fastener in a distal direction into said forceps jaws in said first closed configuration;
(vi) squeezing said jaws into a second closed configuration wherein said jaws are nearer to one another than in said first closed configuration, thereby bringing said fastener into a fully closed postfiring configuration; and
(vii) after the squeezing of said jaws into said second closed configuration, opening said jaws, thereby releasing said fastener.

22. The method in accordance with claim 21 wherein said fastener is disposed in a closed pre-firing configuration in said assembly proximally of said forceps jaws prior to the opening of said jaws and is advanced into said jaws only after the plunging of said jaws into the target tissue, said fastener having legs or prongs that are disposed at a predetermined distance from one another in said closed pre-firing configuration, said legs or prongs being constrained to have a spacing at all times at most as great as said predetermined distance, whereby said fastener is inserted in a substantially closed configuration into the target tissue.

23. The method defined in claim 22 wherein said fastener is constrained by internal stresses to maintain said predetermined distance between said legs or prongs, the squeezing of said jaws inducing a crimping or deformation of said fastener so that said legs or brought into said fully closed postfiring configuration wherein said legs or prongs-are spaced from one another by a distance smaller than said predetermined distance.

24. An endoscopic fastening system comprising an elongate shaft member having an outer diameter sufficiently small to be slidably insertable through an endoscope, said shaft member being provided at a distal end thereof with a pair of forceps jaws, and at a proximal end with an actuation mechanism, a clevis subassembly disposed proximate a proximal aspect of said forceps jaws, said clevis subassembly being operatively coupled with said forceps jaws, said clevis assembly being provided with means to transform a fastener from an open to a closed configuration.

25. The endoscopic fastening system in accordance with claim 24, wherein said clevis subassembly is provided with a forming rail.

26. The endoscopic fastening system in accordance with claim 25, wherein said forming rail is configured for deforming a substantially closed fastener into an open fastener when said fastener traverses along said forming rail in the distal direction.

27. An endoscopic fastening system for securing tissue internal to the body utilizing an endoscope comprising:
an elongate fastener delivery and deployment assembly being provided at a distal end with forceps jaws and at the proximal end with an actuation mechanism for manipulating said jaws between an open and a closed position, one or more surgical fasteners disposed inside said elongate fastener delivery and deployment assembly, said fasteners having a predetermined position relative to said jaws, a pusher rod member provided for advancing said fasteners in a distally directed motion, an inner tubular member configured for housing one or more surgical fasteners disposed in predetermined positions constituting a fastener magazine: and a clevis subassembly operably coupled proximate said forceps jaws, said clevis subassembly being provided with means for opening a partially closed fastener.

28. A method for fastening internal body tissues of a patient, comprising:
(i) inserting an endoscope into a patient's body and is used to visually locate a target tissue to be operated upon;
(ii) inserting a fastener delivery and deployment assembly into a working channel of said endoscope and pushing said assembly in a distal direction towards a target tissue;
(iii) manipulating an actuation mechanism to open forceps jaws of said fastener delivery and deployment assembly and pushing said jaws proximate the target tissue;
(iv) advancing a fastener into said forceps jaws, said fastener being forced into an open position by forming rails;
(v) inserting the open forceps jaws with the open indwelling fastener into the target tissue;
(vi) closing said forceps jaws upon the fastener and the target tissue;
(vii) closing said jaws further to bring the fastener into a fully closed postfiring configuration; and
(viii) opening the forceps jaws to release said fastener after said fastener is brought into said fully closed postfiring configuration, said jaws being removed from the target tissue and extracted from the target tissue.

29. The method in accordance with claim 28, further comprising repeating steps (iii) through (viii) using a plurality of fasteners during a single intubation of the patient.

30. An endoscopic fastening system comprising a delivery and deployment assembly, said assembly including a tubular shaft member, and a spring biased surgical fastener disposed in said shaft member, said fastener being in an unstrained state when in a closed configuration.

31. The endoscopic fastening system in accordance with claim 30, wherein said fastener is configured from a spring biased material taken from the group consisting of plastic, surgical stainless steel, a shape memory material, and Nitinol (NiTi).

32. The endoscopic fastening system in accordance with claim 30, wherein said fastener is provided with a spring member, said spring member, when compressed, effectuates said fastener to assume an opened configuration.

33. The endoscopic fastening system in accordance with claim 32 wherein said spring member is constituted by a proximal bight portion of said fastener joined to legs or prongs of said fastener in a scissors or criss-crossing structure.

34. The endoscopic fastening system, in accordance with claim 30, wherein said shaft member is provided with a hard rigid collar-member having a restrictive lumen for compressing said fastener during a traversing of said lumen by said fastener.

35. The endoscopic fastening system in accordance with claim 34, wherein said restrictive lumen has inner diameter smaller than that of said tubular shaft member.

36. The endoscopic fastening system in accordance with claim 34, wherein said fastener is provided with a spring member, said spring member, when compressed, inducing said fastener to assume an opened configuration, wherein said inner diameter of said restrictive lumen is smaller than said spring member of said fastener.

37. The endoscopic fastening system in accordance with claim 34, wherein said restrictive lumen has a wider proximal opening contiguous with a channel of said tubular shaft member, said opening configured for guiding distal tips of said fastener into said collar member.

38. A surgical fastener provided with two or more prongs and with a spring portion biasing said prongs to a closed configuration, said spring portion being configured to cause said prongs to shift between from the closed configuration to an open configuration upon an application of a transverse compressive force to said spring portion.

39. The fastener in accordance with claim 38 wherein said spring portion is a loop at one end of said fastener, said prongs being continuous with said loop on opposite sides or ends thereof, said loop having a criss-crossed form.

40. A method for fastening internal body tissues of a patient, comprising:
(i) inserting an endoscope through an aperture in a patient's body and using the endoscope to visually locate a target tissue to be operated upon;
(ii) inserting a fastener delivery and deployment assembly into a working channel of an endoscope and pushing the fastener delivery and deployment assembly in the distal direction proximate the target tissue
(iii) operating an actuation means to advance fasteners contained in said fastener delivery and deployment assembly towards a collar member having a restrictive lumen;
(iv) advancing said fastener through said restrictive lumen of said collar member, causing said fastener to assume an open configuration;
(v) pushing the open fastener into target tissue so that said fastener enters said target tissue; and.
(vi) pushing said fastener further in the distal direction until said fastener is released from said restrictive lumen thereby enabling said fastener assume a postfiring closed configuration inside said target tissue.

41. A method for performing a surgical operation on internal body tissues of a patient, comprising:
(i) providing a plurality of surgical fasteners having holes or cavities proximate a proximal end, said fasteners being adjoined to one another by a suture thread, said fasteners being preloaded in pairs or in a magazine fashion;
(ii) applying said fasteners in pairs in organic tissue of a patient;
(iii) synching said suture thread together to provide tissue approximation and/or closure.

42. A surgical fastening instrument with jaws comprising distally rounded tips containing a magazine of staples with similarly rounded tips utilizable, for an example, for ligating tubular structures internal to a patient's body, said fastening system being used in conjunction with an endoscope.
